(19) United States
(12) Patent Application Publication
(10) Pub. No.: US 2021/0386962 A1
(43) Pub. Date: Dec. 16, 2021

(54) WAKEFULNESS MAINTENANCE DEVICE, CONTROL DEVICE, AND PROGRAM

(71) Applicants: KABUSHIKI KAISHA TOKAI RIKA DENKI SEISAKUSHO, Aichi (JP); TOYOTA JIDOSHA KABUSHIKI KAISHA, Aichi-ken (JP)

(72) Inventors: Shuhei TAKEUCHI, Aichi (JP); Aya KADONO, Aichi (JP); Takashi SUZUKI, Aichi (JP); Yoshimasa MIURA, Aichi-ken (JP); Koji IWAKATA, Aichi-ken (JP); Motohiro KOJIMA, Aichi-ken (JP)

(73) Assignees: KABUSHIKI KAISHA TOKAI RIKA DENKI SEISAKUSHO, Aichi (JP); TOYOTA JIDOSHA KABUSHIKI KAISHA, Aichi-ken (JP)

(21) Appl. No.: 17/281,761

(22) PCT Filed: Sep. 26, 2019
(86) PCT No.: PCT/JP2019/037731

§ 371 Date: Mar. 31, 2021

(30) Foreign Application Priority Data
Oct. 18, 2018 (JP) 2018-196986

(51) Int. Cl. A61M 21/00; B60Q 9/00; G08B 21/06; G08B 6/00

(52) U.S. Cl. CPC A61M 21/00; B60Q 9/00; A61M 2021/0083; G08B 6/00; A61M 2021/0022; G08B 21/06

(57) ABSTRACT

[Object] To maintain a wakefulness state of a target more effectively.

[Solution] Provided is an wakefulness maintenance device including: a stimulus provision section configured to provide a vibratory stimulus to at least a part of a body of a target; and a control section configured to control a provision mode and whether or not the stimulus provision section provides the vibratory stimulus, on the basis of an estimated degree of wakefulness of the target. The stimulus provision section includes a contact section configured to come into contact with the part of the body of the target, and a plurality of stimulus generation sections configured to be installed in the contact section and generate the vibratory stimuli under the control of the control section.

### TECHNICAL FIELD

The present invention relates to a wakefulness maintenance device, a control device, and a program.

### BACKGROUND ART

In recent years, devices of monitoring a target and carrying out various kinds of operations on the basis of detected states have been widespread. For example, Patent Literature 1 discloses a technology of maintaining a wakefulness state of a driver by generating a plurality of types of stimuli in the case where drowsiness of the driver is detected.

### CITATION LIST

### Patent Literature

Patent Literature 1: JP 2017-228280A

### DISCLOSURE OF INVENTION

### Technical Problem

The technology disclosed in Patent Literature 1 seeks to maintain the wakefulness state of the driver by providing the plurality of types of stimuli including sound, wind, an aroma, and the like to the driver. However, the stimulus such as the sound, wind, aroma, and the like affects not only the driver but also passengers. Therefore, a state where it is difficult to use such a technology may be envisaged.

In addition, Patent Literature 1 also describes a wakefulness maintenance method using a vibratory stimulus. However, an object of the present invention is to provide a novel and improved wakefulness maintenance device, control device, and program that make it possible to maintain a wakefulness state of a target more effectively.

### Solution to Problem

To solve the above-described problems, according to an aspect of the present invention, there is provided an wakefulness maintenance device including: a stimulus provision section configured to provide a vibratory stimulus to at least a part of a body of a target; and a control section configured to control a provision mode and whether or not the stimulus provision section provides the vibratory stimulus, on the basis of an estimated degree of wakefulness of the target. The stimulus provision section includes a contact section configured to come into contact with the part of the body of the target, and a plurality of stimulus generation sections configured to be installed in the contact section and generate the vibratory stimuli under the control of the control section.

In addition, the control section may cause the plurality of stimulus generation sections to generate the vibratory stimuli in a predetermined order.

In addition, in the case where the degree of wakefulness of the target falls below a threshold, the control section may change a mode of the vibratory stimuli in such a manner that the target has increased sensitivity to the vibratory stimuli.

In addition, in the case where the degree of wakefulness of the target falls below the threshold, the control section may increase drive frequency related to provision of the vibratory stimuli.

In addition, in the case where the degree of wakefulness of the target falls below the threshold, the control section may change at least any of acceleration, a provision interval, provision time, and number of times of provision related to the provision of the vibratory stimuli.

In addition, the wakefulness maintenance device may further include a state detection section configured to estimate a wakefulness state of the target. The state detection section may detect an open/close state of an eyelid of the target on the basis of acquired sensor information, and estimates the degree of wakefulness on the basis of the open/close state.

In addition, to solve the above-described problems, according to another aspect of the present invention, there is provided a control device including a control section configured to control a provision mode and whether or not a stimulus provision section provides a vibratory stimulus to at least a part of a body of a target, on the basis of an estimated degree of wakefulness of the target. The stimulus provision section includes a plurality of stimulus generation sections configured to generate the vibratory stimuli.

In addition, to solve the above-described problems, according to another aspect of the present invention, there is provided a program that causes a computer to function as a control device including a control section configured to control a provision mode and whether or not a stimulus provision section provides a vibratory stimulus to at least a part of a body of a target, on the basis of an estimated degree of wakefulness of the target. The stimulus provision section includes a plurality of stimulus generation sections configured to generate the vibratory stimuli.

### Advantageous Effects of Invention

As described above, according to the present invention, it is possible to maintain a wakefulness state of a target more effectively.

### BRIEF DESCRIPTION OF DRAWINGS

FIG. 1 is a diagram for describing an overview of provision of a tactile stimulus by a wakefulness maintenance device according to an embodiment of the present invention.

FIG. 2 is a diagram for describing an overview of provision of tactile stimuli by the wakefulness maintenance device according to the embodiment.

FIG. 3 is a diagram for describing a configuration example of the wakefulness maintenance device according to the embodiment.

FIG. 4 is a diagram illustrating a configuration example of a palm section according to the embodiment.

FIG. 5 is a diagram illustrating an example of stimulus parameters used for second evaluation according to the embodiment.

### MODE(S) FOR CARRYING OUT THE INVENTION

Hereinafter, preferred embodiments of the present invention will be described in detail with reference to the appended drawings. Note that, in this specification and the appended drawings, structural elements that have substantially the same function and structure are denoted with the same reference signs, and repeated explanation of these structural elements will be omitted.

### 1. Embodiment

### 1.1. Overview

First, an overview of an embodiment of the present invention will be described. For example, as disclosed in Patent Literature 1, the technologies of maintaining a wakefulness state of a target have been proposed in recent years. For example, such technologies may be applied to maintenance of a wakefulness state of a driver who drives various kinds of mobile objects such as a vehicle.

In particular, in recent years, automation of a driving system has been progressing with development of technology. Here, it is recognized that a driver of an autonomous (or semi-autonomous) mobile object tends to relinquish his/her driving tasks. In this case, it is known that the driver who has relinquished his/her driving tasks has a reduced degree of wakefulness.

However, for example, to certainly comply with a take-over request (TOR) from a system, it is necessary not to reduce the degree of wakefulness of a driver in a situation where automated driving and manual driving are combined, which is defined as Level 3 by SAE International.

Here, for example, the technology disclosed in Patent Literature 1 seeks to maintain the wakefulness state of a driver by providing the plurality of types of stimuli including sound, wind, an aroma, vibration, and the like to the driver. However, the stimulus such as the sound, wind, aroma, and the like affects not only the driver but also passengers. Therefore, a state where it is difficult to use such a technology may be envisaged.

In addition, Patent Literature 1 also describes the wakefulness maintenance method using a vibratory stimulus. However, an object of the present invention is to maintain a wakefulness state of a target more effectively.

Therefore, one of features of a wakefulness maintenance device 20 according the embodiment of the present invention is to include a stimulus provision section 230 and a control section 220. The stimulus provision section 230 provides a tactile stimulus to at least a part of a body of a target. The control section 220 controls a provision mode and whether or not the stimulus provision section 230 provides the tactile stimulus, on the basis of an estimated degree of wakefulness of the target.

In addition, one of features of the stimulus provision section 230 according to the present embodiment is to include a contact section 260 and a plurality of stimulus generation sections 270. The contact section 260 comes into contact with the part of the body of the target. The plurality of stimulus generation sections 270 are installed in the contact section 260 and generate tactile stimuli under the control of the control section 220.

Here, the tactile stimuli include a vibratory stimulus for providing a stimulus to the target through vibration, for example. In addition, the above-described provision mode of the tactile stimuli may include a vibration cycle, acceleration, tempo, provision time, the number of times of provision, provision order, or the like, for example.

The wakefulness maintenance device 20 according to the present embodiment not only simply increases intensity of the tactile stimuli, but also changes the provision mode of the tactile stimuli on the basis of the estimated degree of wakefulness of the target. This makes it possible to provide the tactile stimuli in accordance with the degree of wakefulness more effectively, and maintain the wakefulness state of the target more effectively. Note that, hereinafter, a case where the wakefulness maintenance device 20 according to the present embodiment provides vibratory stimuli to the target will be described as a major example.

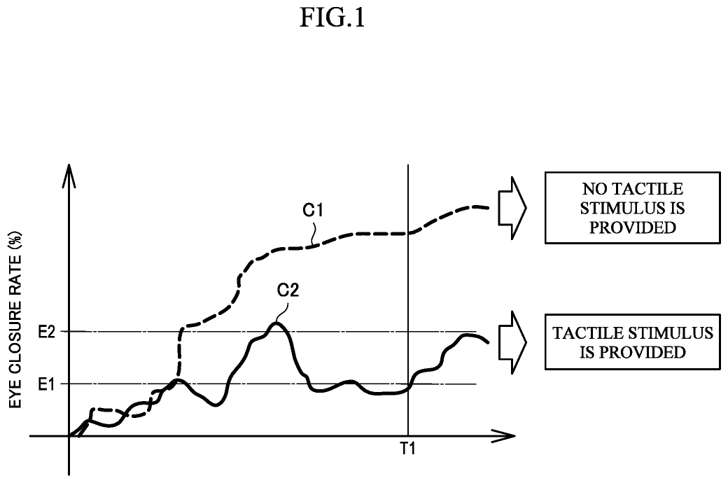

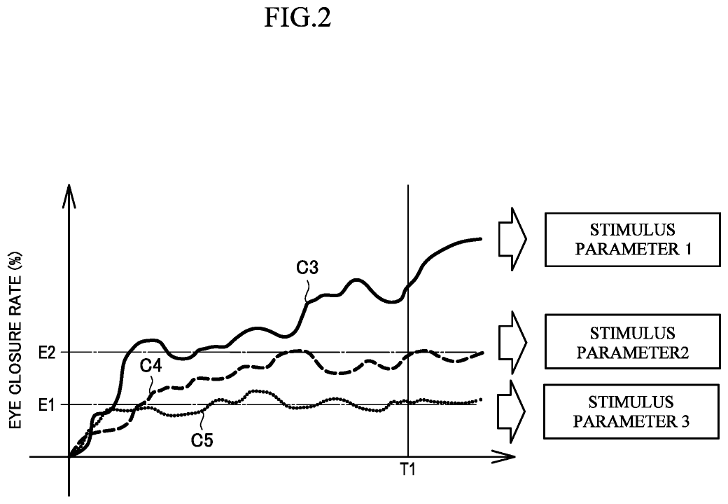

FIG. 1 and FIG. 2 are diagrams for describing an overview of provision of tactile stimuli by the wakefulness maintenance device 20 according to the present embodiment. FIG. 1 and FIG. 2 illustrate a horizontal axis representing a time axis, and a vertical axis representing an eye closure rate, which is an example of an indicator indicating the degree of wakefulness of the target.

Note that, here, the above-described eye closure rate may be a value indicating a rate of a time period during which eyelids of the target are closed within a designated time period, for example. The eye closure rate is closely correlated with the degree of wakefulness, and it is known that the eye closure rate increases with reduction in the degree of wakefulness.

Therefore, the wakefulness maintenance device 20 according to the present embodiment may use an open/close state of the eyelids as an example of the indicator indicating the degree of wakefulness. For example, it is possible for the wakefulness maintenance device 20 according to the present embodiment to estimate (calculate) the degree of wakefulness of the target on the basis of the above-described eye closure rate and an eye opening rate (a value indicating a rate of a time period during which the eyelids of the target is open within the designated time).

For example, the control section 220 of the wakefulness maintenance device 20 according to the present embodiment may control whether or not to provide the vibratory stimuli on the basis of the degree of wakefulness of the target, which has been estimated as described above. For example, FIG. 1 illustrates an eye closure rate transition model (C1) and an eye closure rate transition model (C2). The eye closure rate transition model (C1) is obtained in the case of providing no vibratory stimulus. The eye closure rate transition model (C2) is obtained in the case where the wakefulness maintenance device 20 according to the present embodiment provides a vibratory stimulus when the degree of wakefulness of the target exceeds a threshold E1.

With reference to FIG. 1, in the case of providing no vibratory stimulus, it is predicted that the eye closure rate of the target increases as time elapses, that is, the degree of wakefulness of the target decreases as time elapses. On the other hand, in the case where the wakefulness maintenance device 20 provides the vibratory stimulus, it is expected that the eye closure rate of the target approximately falls within a threshold E2 even after time T1. In other words, it is expected that the wakefulness state of the target is maintained even after the time T1.

Note that, here, the time T1 serves as a maximum value of estimated time it takes to safely stop the mobile object such as the vehicle.

In addition, for example, the wakefulness maintenance device 20 according to the present embodiment may control a provision mode of the vibratory stimulus in accordance with the estimated degree of wakefulness of the target. FIG. 2 illustrates eye closure rate transition models (C3 to C5) obtained in the respective cases of providing the vibratory stimulus to the target by using different stimulus parameters 1 to 3. For example, in the case of C3, provision of a vibratory stimulus is repeated twice by using the stimulus parameter 1 when the eye closure rate of the target reaches the threshold E1. Subsequently, a wakefulness maintenance effect of the stimulus parameter 1 is evaluated. In addition, in the case of C3, the eye closure rate keeps increasing even after the vibratory stimulus is provided as described above. Therefore, next, provision of the vibratory stimulus is repeated four times by using the stimulus parameter 1 after the eye closure rate reaches the threshold E2. Subsequently, the wakefulness maintenance effect is evaluated.

For another example, in a similar way, in the case of C4, provision of a vibratory stimulus is repeated twice by using the stimulus parameter 2 when the eye closure rate of the target reaches the threshold E1, and provision of the vibratory stimulus is repeated four times by using the stimulus parameter 2 when the eye closure rate of the target reaches the threshold E2. In the case of C5, provision of a vibratory stimulus is repeated twice by using the stimulus parameter 3 when the eye closure rate reaches the threshold E1.

As a result, the case of providing the vibratory stimulus in accordance with the stimulus parameter 2 shows that it is possible to maintain the eye closure rate of the target at the threshold E2 or less. In addition, the case of providing the vibratory stimulus in accordance with the stimulus parameter 3 shows that it is possible to approximately maintain the eye closure rate of the target at the threshold E1. As described above, the provision modes of the vibratory stimulus drastically affect the maintenance of wakefulness of the target. Therefore, the wakefulness maintenance device 20 according to the present embodiment is capable of obtaining more effective wakefulness maintenance effects by changing the provision mode of the vibratory stimulus in accordance with the degree of wakefulness of the target.

Note that, in the case of the example illustrated in FIG. 2, the number of times of provision of the stimulus (number of loops) are doubled and the stimulus is provided four times to apply stronger stimuli than the threshold E1 when the eye closure rate reaches the threshold E2, which is higher than the threshold E1. However, intensity of the stimuli may be increased by controlling another element related to the vibratory stimuli. Examples of an element that changes the mode of the vibratory stimulus according to the present embodiment include drive frequency, acceleration, a provision interval, provision time, the number of times of provision, and the like.

In addition, the eye closure rate (eye opening rate) has been described above as an example of the indicator indicating the degree of wakefulness according to the present embodiment. However, the degree of wakefulness according to the present embodiment may be estimated on the basis of any of heart rate, nose skin temperature, breathing variation, and facial expressions including a yawn, or a combination thereof, for example. Next, functions of the wakefulness maintenance device 20 according to the present embodiment and effects achieved by the functions will be described in detail.

### 1.2 Configuration Example of Wakefulness Maintenance Device 20

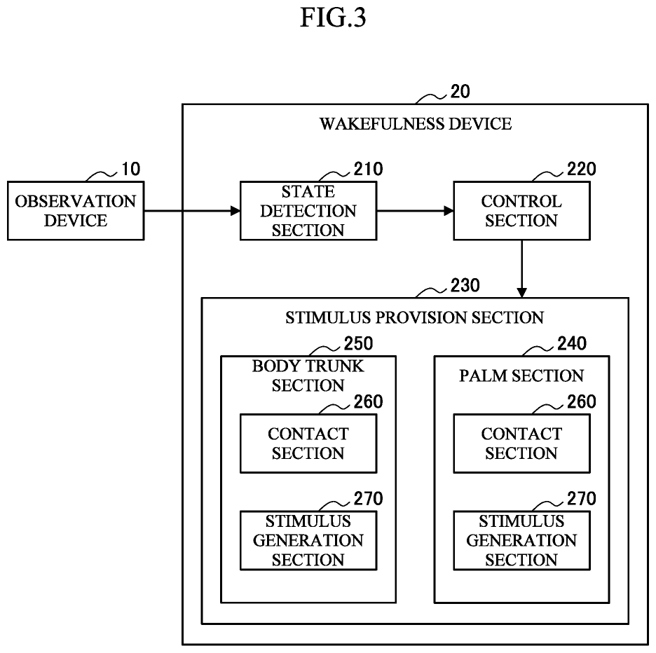

First, a configuration example of the wakefulness maintenance device 20 according to the present embodiment will be described. FIG. 3 is a diagram for describing the configuration example of the wakefulness maintenance device 20 according to the present embodiment. With reference to FIG. 3, the wakefulness maintenance device 20 according to the present embodiment includes a state detection section 210, the control section 220, and the stimulus provision section 230.

(State Detection Section 210)

The state detection section 210 according to the present embodiment has a function of estimating the degree of wakefulness of the target on the basis of various kinds of sensor information acquired by an observation device 10. Note that, the observation device 10 according to the present embodiment is a device that continuously monitors various states of the target. For example, the state detection section 210 according to the present embodiment may detect the open/close state of the eyelids of the target on the basis of an image of the target captured by the observation device 10, and may estimate the degree of wakefulness on the basis of the open/close state. For example, the state detection section 210 according to the present embodiment may calculate the eye closure rate, the eye opening rate, or the like on the basis of the image of the target, and may use the calculated rate as the degree of wakefulness.

In addition, as described above, the state detection section 210 according to the present embodiment may estimate the degree of wakefulness of the target on the basis of the heart rate, the skin temperature, or the like. Therefore, the observation device 10 according to the present embodiment may include at least one of a camera, a laser, an optical sensor, a temperature sensor, a pulse sensor, and the like, for example.

(Control Section 220)

The control section 220 according to the present embodiment has a function of controlling the provision mode and whether or not the stimulus provision section 230 provides the vibratory stimulus on the basis of the degree of wakefulness of the target, which has been estimated by the state detection section 210.

For example, in the case where the degree of wakefulness of the target falls below a threshold, the control section 220 according to the present embodiment may change the mode of the vibratory stimulus in such a manner that the target has increased sensitivity to the vibratory stimulus. At this time, the control section 220 according to the present embodiment may increase the sensitivity by increasing the drive frequency, acceleration, or the like. In addition, the control section 220 may increase the sensitivity by increasing the number of times of providing the vibratory stimulus, extending the provision time, or shortening the provision interval.

For example, the control section 220 according to the present embodiment may be implemented by one, two, or more processors. Examples of the processors include a central processing unit (CPU), a microcontroller unit (MCU), and the like.

(Stimulus Provision Section 230)

The stimulus provision section 230 according to the present embodiment has a function of providing the vibratory stimulus to the target under the control of the control section 220. The stimulus provision section 230 according to the present embodiment may include a plurality of stimulus provision parts corresponding to parts of the body of the target. For example, the stimulus provision section 230 may include a palm section 240, a body trunk section 250, and the like. The palm section 240 provides a vibratory stimulus to a palm of the target. The body trunk section 250 provides a vibratory stimulus to an arm (from a hand to a shoulder), a back, a waist, a hip, a thigh, or the like of the target.

In addition, each of the palm section 240 and the body trunk section 250 includes the contact section 260 and the stimulus generation sections 270. The contact section 260 comes into contact with a part of the body of the target. The stimulus generation sections 270 is installed in the contact section 260 and generates the vibratory stimulus under the control of the control section 220.

For example, the stimulus provision section 230 according to the present embodiment may be installed in an autonomous driving assistance device, which is installed in a vehicle interior of the mobile object having autonomous driving functions. Here, the above-described autonomous driving assistance device may be a device that provides the vibratory stimuli to the target to maintain the wakefulness state of the target who has relinquished his/her driving tasks.

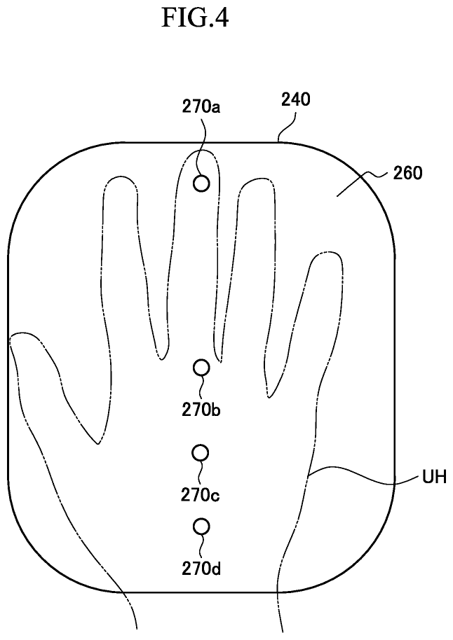

FIG. 4 is a diagram illustrating a configuration example of the palm section 240 according to the present embodiment. For example, the palm section 240 according to the present embodiment may be installed in the autonomous assistance device, which is installed in the vehicle interior of the mobile object having the autonomous driving functions. Here, the autonomous driving assistance device is a device operated by the driver when the driver suddenly needs to take over driving operation during autonomous driving. The driving assistance device may be installed independently from a steering wheel. For example, in the case where the system intends to take an evasive action to swerve to the right on the basis of detection of an obstacle in a direction in which the mobile object is going but the driver determines to take an evasive action to swerve to the left, the driver may assist the autonomous driving by performing an operation of tilting the autonomous driving assistance device to the left.

To achieve the above-described operation, the autonomous driving assistance device according to the present embodiment may have a shape that can be held by a hand of the driver, that is, a hand of the target, or a shape on which a palm of the target can be placed. FIG. 4 illustrates an example in which the contact section 260 of the palm section 240 installed in the autonomous driving assistance device has a shape and size that allow an operator to place his/her hand UH on the contact section 260. For example, the contact section 260 of the palm section 240 may be formed by using resin material or the like such as polycarbonates, in such a manner that the contact section 260 has rigidity that makes it possible to instruct the hand UH.

In addition, the contact section 260 of the palm section 240 may have a curved surface such as an arc-like surface that makes it easier for the operator to place his/her hand UH on the contact section 260. Note that, the shape of the contact section 260 can be freely designed. The shape of the contact section 260 may be a combination of flat surfaces and curved surfaces.

In addition, in the case of the example illustrated in FIG. 4, the contact section 260 of the palm section 240 includes four stimulus generation sections 270a to 270d that are arrayed from a tip a finger of the hand UH to a wrist. As described above, the stimulus provision section 230 according to the present embodiment may include the plurality of stimulus provision sections 270.

In this case, the control section 220 according to the present embodiment can cause the plurality of stimulus generation sections 270 to generate vibratory stimuli in a predetermined order. For example, in the case of the example illustrated in FIG. 4, the control section 220 may cause the stimulus generation sections 270a, 270b, 270c, and 270d to generate the vibratory stimuli in this order. Under such control, it is possible to give the vibratory stimuli fluently in order from an end of the body in such a manner that the vibratory stimuli pass through the tip of the finger, the base of the finger, the center of the palm, and the heel of the hand in this order, for example. This allows the target to perceive illusionary motion.

Here, wording “illusionary motion” according to the present embodiment means a phenomenon of feeling as if motion were made although the motion is not made in practice. Such illusionary motion is made when the stimuli are sequentially given to a plurality of parts of the body. For example, the target may perceive the illusionary motion between the tip of the finger and the base of the finger in the case where the stimulus generation section 270a instantaneously provides a vibratory stimulus to the tip of the finger and then the stimulus generation section 270b provides a vibratory stimulus to the base of the finger. In a similar way, in the case where the stimulus generation sections 270c and 270d provide vibratory stimuli sequentially, the target may perceive illusionary motion as if the illusionary motion were made on the whole hand in such a manner that the illusionary motion moves from the tip of the finger to the heel of the hand.

As described above, the wakefulness maintenance device 20 according to the present embodiment sequentially provides the vibratory stimuli to the plurality of parts of the palm or the like. This allows the target to perceive the illusionary motion and it is possible to achieve a stronger wakefulness maintenance effect.

Note that, FIG. 4 illustrates the example in which the palm section 240 includes the four stimulus generation sections 270a to 270d. However, the number of stimulus generation sections 270 according to the present embodiment is not limited thereto. It is sufficient that the two or more stimulus generation sections 270 according to the present embodiment are prepared. In the case where the two or more stimulus generation sections 270 are prepared, it is possible for the target to perceive the above-described illusionary motion. Alternatively, in the case where the three or more stimulus generation sections 270 are prepared, it is possible for the target to perceive the movement direction of the illusionary motion. Alternatively, in the case where the four or more stimulus generation sections 270 are prepared, at least three of the stimulus generation sections 270 are in contact with a hand of the target even if the hand is small. Therefore, it is expected to obtain the wakefulness maintenance effect more certainly. In addition, the array of the plurality of stimulus generation sections 270 are not limited to the linear arrangement illustrated in FIG. 4. The plurality of stimulus generation sections 270 may be arranged non-linearly.

In addition, the control section 220 according to the present embodiment may cause the plurality of stimulus generation sections 270 installed in the palm section 240 or the body trunk section 250 to generate the vibratory stimuli in a predetermined order. For example, it is possible for the control section 220 according to the present embodiment to control the stimulus provision section 230 in such a manner that the vibratory stimuli are sequentially provided along a body axis starting from a hand of the target. Here, the body axis is various kinds of axes indicating the direction of the body such as a cephalocaudal axis, a dorsoventral axis, or a left-right axis.

For example, in the case where the body trunk section 250 includes the stimulus generation sections 270 at positions corresponding to the back and the hip of the target, the control section 220 may cause the stimulus generation section 270 of the palm section 240, the stimulus generation section 270 of the body trunk section 250 corresponding to the back, and the stimulus generation section 270 of the body trunk section 250 corresponding to the hip to generate the vibratory stimuli in this order.

As described above, the control section 220 performs control in such a manner that the vibratory stimuli are sequentially provided along the body axis of the target. This allows the target to easily perceive the vibratory stimuli and allows the target to feel as if the motion was made. In other words, this allows the target to perceive the illusionary motion. Accordingly, it is expected to obtain the effect of maintaining the wakefulness state of the target more effectively.

The configuration example of the wakefulness maintenance device 20 according to the present embodiment has been described above in detail. Note that, the configuration described above with reference to FIG. 3 and FIG. 4 is a mere example. The configuration of the wakefulness maintenance device 20 according to the present embodiment is not limited thereto.

For example, the state detection section 210, the control section 220, and the stimulus provision section 230 according to the present embodiment may be implemented as respective functions of different devices. For example, the control section 220 may be independently configured as a control device, and may control a stimulus provision device on the basis of the degree of wakefulness of the target, which is acquired via a network. The stimulus provision device is prepared as another device. The configuration of the wakefulness maintenance device 20 according to the present embodiment can be flexibly modified in accordance with specifications and operations.

### 1.3. Evaluation

Next, results of evaluating stimulus provision methods according to the present embodiment will be described. Here, two types of evaluations are performed to verify effects achieved by the wakefulness maintenance device 20 according to the present embodiment.

Note that, the both evaluations were performed on the assumption of the above-described Level 3 of autonomous driving (in order words, a state where it is not necessary for a driver to monitor surroundings but the driver has to keep awake to prepare for the TOR). Inspections were conducted for 30 minutes in such a manner that a vehicle driving video and vehicle driving sound are reproduced in a darkroom to replicate an actual vehicle interior.

Note that, an examinee was told that this is driving simulation on the assumption of Level 3 of autonomous driving, and it is not necessary for the examinee to monitor surroundings but the examinee has to keep awake to prepare for the TOR.

In addition, the above-described eye closure rate is used as an indicator of the degree of wakefulness.

First, in the case of the first evaluation, the plurality of stimulus generation sections 270 of the palm section 240 and the plurality of stimulus generation sections 270 of the body trunk section 250 generate vibratory stimuli sequentially. More specifically, the vibratory stimuli are given fluently to a hand, back, waist, hip, and thigh of the target in this order, and transition of the wakefulness state is observed.

In addition, in the case of the first evaluation, three sets of conditions for stimulus parameters are set, and are compared with a case where no stimulus is given.

First, in a “standard” mode, acceleration of the palm section 240 is set to 5.3 G and acceleration of the body trunk section 250 is set to 1.8 G, frequency of the palm section 240 is set to 45 Hz, and frequency of the body trunk section 250 is set to 50 Hz. In addition, vibration time is set to 1.5 s, and a provision interval is set to 0.75 s. In addition, the number of times of vibration is set to one. When the eye closure rate exceeds the threshold E1, the vibratory stimulus is generated on the basis of the above-described stimulus parameters.

In addition to the “standard” mode, a “large acceleration” mode, and a “short vibration time” mode are compared. In the “large acceleration” mode, acceleration of a vibratory stimulus generated by the body trunk section 250 is set to twice the acceleration in the “standard” mode. In the “short vibration time” mode, each of the vibration time and the provision interval is set to a half of the vibration time or the provision interval in the “standard” mode, and the number of times of vibration is set to two.

As a result of performing evaluations under the above-described conditions, gradual increase in the eye closure rate is observed since start of the evaluation in the case where no stimulus is given. However, in the case where a vibratory stimulus is given, it is observed that the examinee keeps awake for a while after the start of evaluation in any of the “standard” mode, the “large acceleration” mode, and the “short vibration time” mode.

More specifically, in the “standard” mode and the “large acceleration” mode, the wakefulness maintenance effect is observed approximately with regard to 56% of the examinees. In the “short vibration time” mode, the wakefulness maintenance effect is observed approximately with regard to 45% of the examinees.

The above-described examination results show that when the wakefulness maintenance device 20 according to the present embodiment provides the vibratory stimulus, it is possible to maintain the wakefulness state of the target longer than the case where no vibratory stimulus is given.

Next, second evaluation according to the present embodiment will be described. In the case of the second evaluation according to the present embodiment, the vibratory stimulus is provided to a palm in a concentrative manner and transition of the wakefulness state of the examinee is observed. The palms are known as parts which are more sensitive to the stimulus than the back, the waist, and the like. In other words, in the case of the second evaluation according to the present embodiment, only the palm section 240 generates the vibratory stimulus, and the wakefulness maintenance effect is verified.

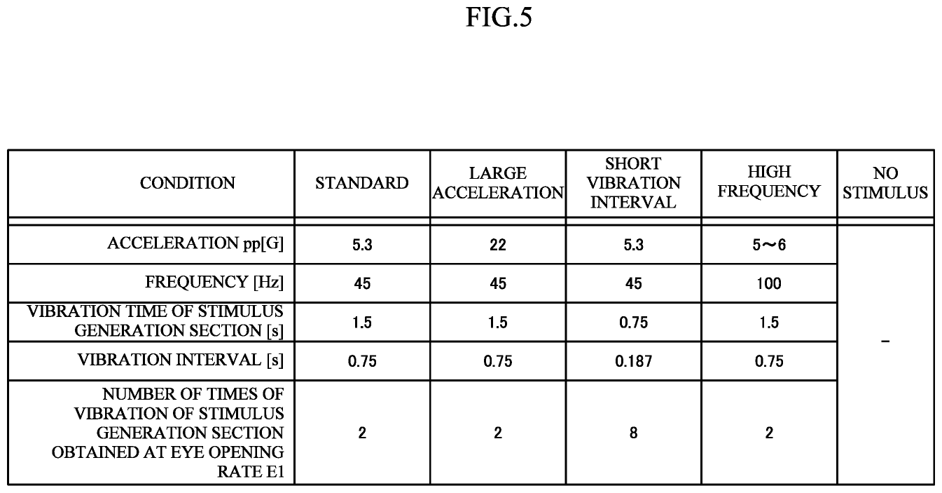

Note that, in the case of the second evaluation, four sets of conditions for stimulus parameters are set as illustrated in FIG. 5. FIG. 5 is a diagram illustrating the stimulus parameters used for the second evaluation according to the present embodiment.

First, in a “standard” mode, acceleration of a vibratory stimulus provided by the palm section 240 is set to 5.3 G, and frequency of the vibratory stimulus provided by the palm section 240 is set to 45 Hz. In addition, vibration time of the stimulus generation section 270 is set to 1.5 s, and a provision interval is set to 0.75 s. In addition, the number of times of vibration of the stimulus generation section 270 is set to two. When the eye closure rate exceeds the threshold E1, the vibratory stimulus is generated on the basis of the above-described stimulus parameters. Note that, the above-described number of times of vibration of the stimulus generation section 270 means the number of times of sequentially vibrating the plurality of stimulus generation sections 270 of the palm section 240. For example, when the palm section 240 includes the four stimulus generation sections 270a to 270d as illustrated in FIG. 4, the case where the number of times of vibration is two means two repetitions of a loop in which the stimulus generation sections 270a to 570d generate the vibratory stimulus sequentially.

In addition, in the “large acceleration” mode, acceleration of the vibratory stimulus provided by the palm section 240 is set to 22 G, which is the maximum acceleration to be achievable on the basis of performance.

In addition, the vibration time and the provision interval in the “short vibration time” mode are set to ¼ of the vibration time and the provision interval in the “standard” mode. In addition, the number of times of vibration in the “short vibration time” mode is set to 8.

In addition, in the case of a “high frequency” mode, frequency of the vibratory stimulus provided by the palm section 240 is set to 100 Hz, which is about twice of the frequency in the “standard” mode.

As a result of performing evaluations under the above-described conditions, gradual increase in the eye closure rate is observed since start of the evaluation in the case where no stimulus is given. However, in the “standard” mode, the “large acceleration” mode, and the “short vibration time” mode, it is observed that the eye closure rate is approximately equal to or less than the threshold E2 until the evaluation ends.

In addition, in the “high frequency” mode, the eye closure rate hardly increases unlike the above-described three set of conditions, and the wakefulness state of the examinee is maintained until the evaluation ends.

More specifically, in the “standard” mode, the “large acceleration” mode, and the “short vibration time” mode, the wakefulness maintenance effect is observed approximately with regard to 60% of the examinees. In the “high frequency” mode, the wakefulness maintenance effect is observed approximately with regard to 80% of the examinees.

The above-described evaluation results show that the wakefulness maintenance effect increases when the stimulus is repeatedly provided to the palm, which is more sensitive to the stimulus than the body trunk. Note that, no difference in effect between the “standard” mode and the “short vibration time” mode is observed because the examinee could possibly get used to the vibratory stimulus due to increase in the number of times of repetition or decrease in the effect caused by shortening the vibration time could possibly counteract increase in the effect caused by increasing the number of times repetition.

In addition, the above-described evaluation results show that the wakefulness maintenance effect increases more with the increase in frequency. Here, frequency of 100 Hz in the “high frequency” mode is known as a frequency band to which Pacinian corpuscles in dermis sensitively react. This suggests a possibility that the frequency band to which Pacinian corpuscles sensitively reacts is effective in keeping awake. Note that, the frequency of 45 Hz used in the “standard” mode and the like is a frequency band to which Meissner's corpuscles in epidermis sensitively react.

Therefore, for example, in the case where the degree of wakefulness of the target falls below a threshold, the control section 220 according to the present embodiment may increase the drive frequency for providing the vibratory stimulus and set the drive frequency to the frequency band to which Pacinian corpuscles sensitively react. Under the control of the control section 220 according to the present embodiment as described above, it is expected to maintain the wakefulness state of the target more effectively.

Note that, the human body has different skin thicknesses at different parts. Therefore, it is also assumed that Pacinian corpuscles at the different parts of the body sensitively react to different frequency bands. Therefore, the control section 220 according to the present embodiment may set frequencies for respective parts of the body corresponding to the stimulus generations sections 270 in such a manner that the frequencies are customized for the respective parts of the body and Pacinian corpuscles at the respective parts of the body sensitively reacts to the frequencies.

The results of evaluating the stimulus provision methods used by the wakefulness maintenance device 20 according to the present embodiment have been described above. As described above, the wakefulness maintenance device 20 according to the present embodiment sequentially provides the vibratory stimuli to the plurality of parts of the palm. This allows the target to perceive the illusionary motion and it is possible to maintain the wakefulness state of the target more effectively.

Note that, the example in which the control section 220 compares the degree of wakefulness of the target with the thresholds and controls the provision mode and whether or not to provide the vibratory stimulus, has been described above as the major example. However, the control of the control section 220 according to the present embodiment is not limited thereto.

For example, the control section 220 according to the present embodiment may change the provision mode in accordance with the number of times of providing the vibratory stimulus to mitigate the effect of habituation of the vibratory stimulus. In addition, the control section 220 according to the present embodiment may change the provision mode of the vibratory stimulus if the degree of wakefulness is not changed after provision of the vibratory stimulus.

In addition, the control section 220 according to the present embodiment may change the provision mode of the vibratory stimulus on the basis of an external factor. For example, the control section 220 may provide the vibratory stimulus in different modes between nighttime and daytime. In the nighttime, it is generally assumed that the degree of wakefulness easily decreases. In the daytime, it is generally assumed that the degree of wakefulness does not tend to decrease. It is possible for the control section 220 according to the present embodiment to combine the above-described conditions and provide the vibratory stimulus in various kinds of modes.

### 2 Conclusion

As described above, one of features of the wakefulness maintenance device 20 according an embodiment of the present invention is to include the stimulus provision section 230 and the control section 220. The stimulus provision section 230 provides a tactile stimulus to a palm of a target. The control section 220 controls a provision mode and whether or not the stimulus provision section 230 provides the tactile stimulus, on the basis of an estimated degree of wakefulness of the target. In addition, one of features of the stimulus provision section 230 according to an embodiment of the present invention is to include the contact section 260 and the plurality of stimulus generation sections 270. The contact section 260 comes into contact with a part of the body of the target. The plurality of stimulus generation sections 270 are installed in the contact section 260 and generate tactile stimuli under the control of the control section 220. In addition, one of the features of the stimulus provision section 230 according to an embodiment of the present invention is to be held by a hand of the target or to be installed in the autonomous driving assistance device, on which the palm of the target can be placed. Such a configuration makes it possible to maintain the wakefulness state of the target more effectively.

The preferred embodiments of the present invention have been described above in detail with reference to the accompanying drawings, whilst the present invention is not limited to the above examples. A person skilled in the art of the present invention may find various alternations and modifications within the scope of the appended claims, and it should be understood that they will naturally come under the technical scope of the present invention.

In addition, the effects described herein are illustrative or exemplary but not limitative. That is, besides the above effects or instead of the above effects, the technology according to the present disclosure may provide other effects that are obvious to a person skilled in the art.

In addition, a program for causing hardware such as a CPU, a ROM, or a RAM installed in a computer to exert a function equal to that of the configuration of the control section 220 can be created. Also, a non-transitory computer readable recording medium having the program recorded therein may be provided.

### REFERENCE SIGNS LIST

10 observation device
20 wakefulness maintenance device
210 state detection section
220 control section
230 stimulus provision section
240 palm section
250 body trunk section
260 contact section
270 stimulus generation section

1. An wakefulness maintenance device comprising:
a stimulus provision section configured to provide a vibratory stimulus to at least a part of a body of a target; and
a control section configured to control a provision mode and whether or not the stimulus provision section provides the vibratory stimulus, on a basis of an estimated degree of wakefulness of the target,
wherein the stimulus provision section includes
a contact section configured to come into contact with the part of the body of the target, and
a plurality of stimulus generation sections configured to be installed in the contact section and generate the vibratory stimuli under the control of the control section.

2. The wakefulness maintenance device according to claim 1,
wherein the control section causes the plurality of stimulus generation sections to generate the vibratory stimuli in a predetermined order.

3. The wakefulness maintenance device according to claim 1,
wherein, in a case where the degree of wakefulness of the target falls below a threshold, the control section changes a mode of the vibratory stimuli in such a manner that the target has increased sensitivity to the vibratory stimuli.

4. The wakefulness maintenance device according to claim 3,
wherein, in a case where the degree of wakefulness of the target falls below the threshold, the control section increases drive frequency related to provision of the vibratory stimuli.

5. The wakefulness maintenance device according to claim 3,
wherein, in a case where the degree of wakefulness of the target falls below the threshold, the control section changes at least any of acceleration, a provision interval, provision time, and number of times of provision related to the provision of the vibratory stimuli.

6. The wakefulness maintenance device according to claim 1, further comprising
a state detection section configured to estimate a wakefulness state of the target,
wherein the state detection section detects an open/close state of an eyelid of the target on a basis of acquired sensor information, and estimates the degree of wakefulness on a basis of the open/close state.

7. A control device comprising
a control section configured to control a provision mode and whether or not a stimulus provision section provides a vibratory stimulus to at least a part of a body of a target, on a basis of an estimated degree of wakefulness of the target,
wherein the stimulus provision section includes a plurality of stimulus generation sections configured to generate the vibratory stimuli.

8. A non-transitory storage medium that stores a program causing a computer to function as
a control device including
a control section configured to control a provision mode and whether or not a stimulus provision section provides a vibratory stimulus to at least a part of a body of a target, on a basis of an estimated degree of wakefulness of the target,
wherein the stimulus provision section includes a plurality of stimulus generation sections configured to generate the vibratory stimuli.
